(19) United States
(12) Patent Application Publication
(10) Pub. No.: US 2017/0009242 A1
(43) Pub. Date: Jan. 12, 2017

(54) CRISPR-Mediated Genome Engineering for Protein Depletion

(71) Applicants: Whitehead Institute for Biomedical Research, Cambridge, MA (US); The Johns Hopkins University, Baltimore, MD (US)

(72) Inventors: Kara McKinley, Cambridge, MA (US); Iain Cheeseman, Cambridge, MA (US); Andrew Holland, Baltimore, MD (US)

(21) Appl. No.: 15/203,415

(22) Filed: Jul. 6, 2016

Related U.S. Application Data
(60) Provisional application No. 62/189,198, filed on Jul. 6, 2015.
(60) Provisional application No. 62/196,026, filed on Jul. 23, 2015.

(51) Int. Cl. C12N 15/63; C12N 15/90; C07K 14/415; C12N 9/22

(52) U.S. Cl. CPC C12N 15/635; C12N 9/22; C12Y 301/00; C12N 15/907; C07K 14/415; C12N 2800/80; C07K 2319/95

(57) ABSTRACT

The present invention provides compositions and methods for tagging a target gene with a degron (e.g., auxin-inducible degron) in a variety of eukaryotic cells using the CRISPR genome-editing technology. Also provided are cells that have been genetically modified using such compositions and methods.

### RELATED APPLICATION

This application claims the benefit of U.S. Provisional Application No. 62/189,198, filed on Jul. 6, 2015 and U.S. Provisional Application No. 62/196,026, filed Jul. 23, 2015. The entire teachings of the above application are incorporated herein by reference.

### GOVERNMENT SUPPORT

This invention was made with government support under GM088313 and GM114119 from the National Institutes of Health. The government has certain rights in the invention.

### INCORPORATION BY REFERENCE OF MATERIAL IN ASCII TEXT FILE

This application incorporates by reference the Sequence Listing contained in the following ASCII text file being submitted concurrently herewith:
a) File name: 03992058002SEQUENCELISTING.txt; created Jul. 6, 2016, 2 KB in size.

### BACKGROUND OF THE INVENTION

Cellular functions are carried out through a complex network of small molecules and macromolecules, such as DNA, RNA, and proteins. Proteins are the primary drivers behind the majority of cellular functions. Methods to understand how a specific protein functions generally include strategies to remove the particular protein from the system, and subsequently observe how the system changes. Such strategies are at the core of biological research, and have proven to be powerful in understanding protein function.

In many organisms, a particular protein can be removed from a system by disrupting the gene that encodes the protein (e.g., generating a mutation), or by suppressing it at the mRNA level (e.g., RNA interference, also known as RNAi). In mammalian cells, the latter has been the predominant strategy to deplete proteins due to the lack of genome engineering technologies. However, such methods have several limitations. For example, RNAi can be challenging to execute with high penetrance, and must be carefully controlled to eliminate the possibility of off-target effects. Significantly, the protein of interest is not directly eliminated; rather, the process simply halts the production of more protein. Thus, the lifetime of the existing protein in the cell must be accounted for before the cellular function can be analyzed. Some proteins are highly stable, causing this to be time consuming, and defects may accumulate while waiting for the protein to be fully depleted. Moreover, once the production of a particular protein has been suppressed, it cannot be readily re-expressed in the system for further functional studies of the protein.

The auxin-inducible degron (AID) system provides a solution to some of the challenges inherent in methods that suppress protein production at the mRNA level by targeting specific proteins for rapid degradation (Nishimura et al., Nature Methods 6(12):917-22, 2009). In brief, a protein of interest is tagged with an AID and expressed in cells containing a plant subunit of the SCF complex, TIR1. When the plant hormone auxin is introduced into the system, the tagged protein is targeted for degradation. This allows a protein to be rapidly depleted at the level of existing protein, rather than blocking the synthesis of new protein. However, it has not been possible to fully exploit these advantages in mammalian cell culture because the method often requires coupling with existing strategies to block protein synthesis (e.g. RNAi). Due to challenges in human genome engineering, an AID-tagged protein must often be overexpressed as a transgene and the endogenous gene product suppressed by, e.g., RNAi. RNAi-based methods are often unable to completely deplete proteins of interest and suffer from unwanted off-target effects. Thus, existing auxin strategies present of the same challenges that hinder traditional gene suppression techniques.

Accordingly, there is a significant unmet need for a method to achieve rapid depletion of an endogenous protein without suppressing endogenous protein production at the mRNA level.

### SUMMARY OF THE INVENTION

The present invention overcomes some of the difficulties associated with using the auxin-inducible degron (AID) system in cells where homologous recombination is inefficient and difficult by employing the CRISPR genome-editing technology.

Thus, in one aspect, the present invention provides a method of tagging a target gene in a cell with a nucleotide sequence encoding an AID. The method comprises introducing into a cell a nucleic acid comprising a nucleotide sequence encoding a synthetic guide ribonucleic acid (sgRNA), wherein the sgRNA is complementary to a target nucleotide sequence in or near a target gene. The method further comprises introducing into a cell a nucleic acid comprising a nucleotide sequence encoding a clustered regularly interspaced short palindromic repeat (CRISPR)-associated nuclease 9 (Cas9). The method also includes introducing into the cell a repair template comprising a nucleotide sequence encoding an AID. The sgRNA and Cas9 nuclease are expressed in the presence of the repair template in the cell, thereby allowing homologous-recombination of a targeted double strand break and subsequent tagging of the target gene with the nucleotide sequence encoding the AID.

In a related aspect, the present invention also provides a genetically-modified cell comprising a gene tagged with a nucleotide sequence encoding an AID produced according to the method of the invention.

The present invention also provides, in certain aspects, a genetically-modified cell comprising a nucleic acid comprising a nucleotide sequence encoding a Cas9 and a nucleic acid comprising a nucleotide sequence encoding a transport inhibitor response 1 (TIR1) receptor.

In other aspects, the present invention provides a nucleic acid comprising a repair template having a nucleotide sequence encoding an AID.

As described herein, the present invention utilizes CRISPR genome-editing technology and the AID system to provide compositions and methods for rapidly and reversibly depleting a protein of interest in a cell. The invention allows for the effective removal of a protein with ensuing phenotype in minimal time with high penetrance and temporal resolution, while avoiding off-target effects on non-AID tagged genes. In addition, the methods of the invention do not require coupling with other methods of suppressing protein production to achieve effective degradation of the target protein.

### BRIEF DESCRIPTION OF THE DRAWINGS

The foregoing will be apparent from the following more particular description of example embodiments of the invention, as illustrated in the accompanying drawings.

FIG. 1 generally depicts the auxin-inducible degron system. The protein of interest (target) is tagged with an auxin-inducible degron (AID), and the TIR1 subunit of the SCF (Skp1, Cull, F-box) complex from the rice Oryza sativa (osTIR1) is expressed as a transgene. Binding of auxin family hormones, such as Indole-3-acetic acid (IAA) promotes interaction between TIR1 and the degron-tagged protein. This in turn recruits an E2 ubiquitin ligase to poly-ubiquitinate the AID-tagged protein and targets the protein for degradation by the 26S proteasome.

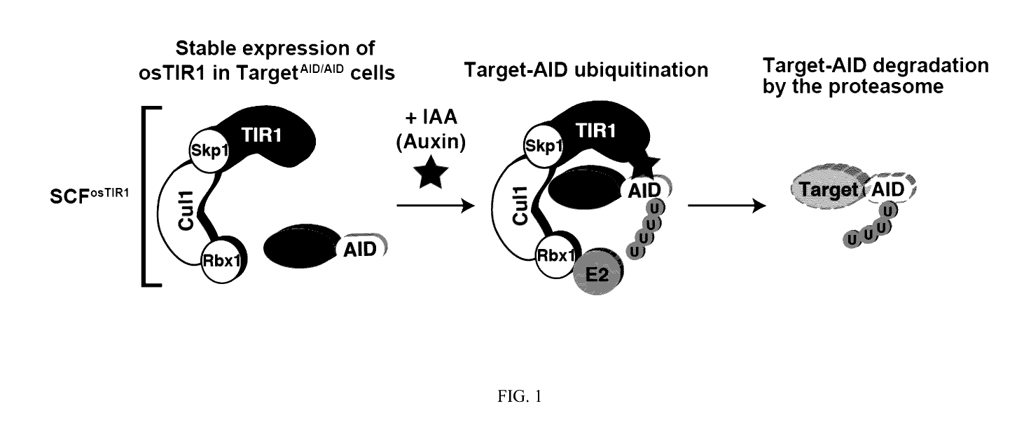

FIGS. 2A and 2B illustrate tagging of endogenous loci with a sequence encoding AID-EGFP at the N- or C-terminus of a target protein. For C-terminal tagging (FIG. 2A), the rescue construct (the lower construct in FIG. 2A) modifies the 3′ exon with a sequence encoding a linker and the AID-EGFP sequence, followed by a foxed neomycin resistance gene. For N-terminal tagging (FIG. 2B), the rescue construct (the lower construct in FIG. 2B) modifies the 5′ exon with a sequence encoding a linker and the EGFP-AID sequence. In either instance, to prevent re-cutting of the repaired allele by Cas9, the PAM sequence (e.g., NGG) can be mutated in the rescue construct (e.g., NTT), or the sgRNA binding site can be selected such that it is disrupted by insertion of the AID-EGFP sequence.

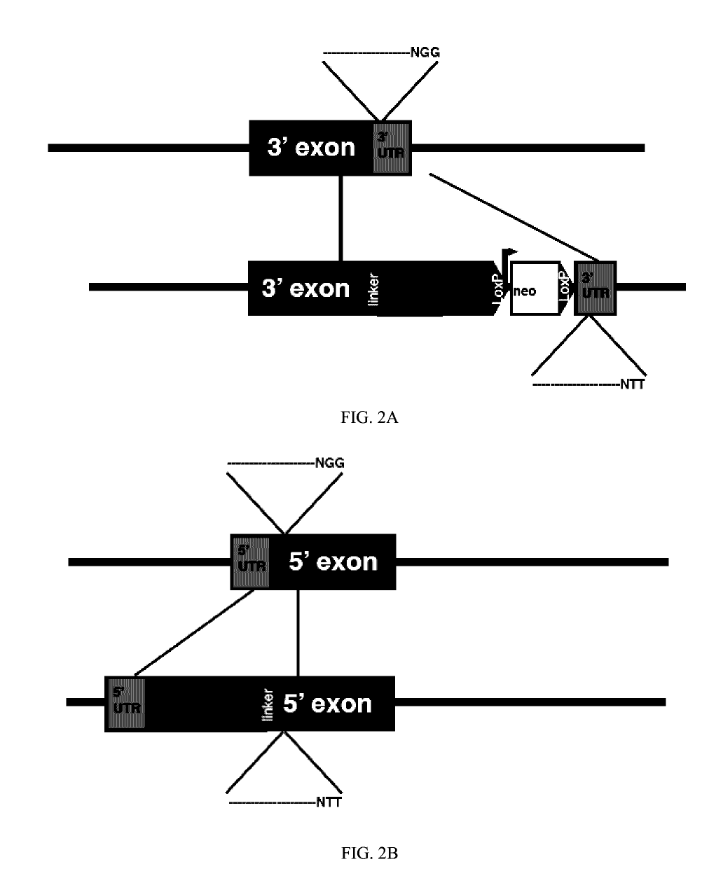

FIG. 3 illustrates the results of endogenous tagging of centromere protein I (CENP-I) with AID-EGFP using CRISPR. Immunofluorescence images show the localization of the CENP-I-AID-EGFP fusion protein to kinetochores in an interphase cell in the absence of IAA. Upon addition of IAA, the fusion protein is no longer observed and associated proteins such as CENP-T are also lost from kinetochores. Scale bar=5 μm.

### DETAILED DESCRIPTION OF THE INVENTION

A description of example embodiments of the invention follows.

Methods of depleting a target protein by exploiting specific protein degradation pathways have been described (Zhou, Curr. Opin. Chem. Biol. 9:51-55, 2005; Banaszynski and Wandless, Chem. Biol. 13:11-21, 2006; Holland et al., PNAS 109(49):E3350-57, 2012; Lambrus et al., J. Cell Biol. 210:63-77, 2015). For example, the auxin-inducible degron (AID) system, which originates from plants, is a powerful tool to conditionally deplete protein levels (Nishimura et al., Nature Methods 6(12):917-22, 2009). Auxin represents a family of plant hormones that control gene expression during many aspects of growth and development (Teale et al., Nat. Rev. Mol. Cell Biol. 7:847:859 (2006)). Auxin family hormones, such as the naturally-occurring indole-3-acetic acid (IAA) and the synthetic 1-naphthaleneacetic acid (NAA), bind to the F-box transport inhibitor response 1 (TIR1) protein and promote the interaction of the E3 ubiquitin ligase SCF-TIR1 (a form of Skp1, Cullin and F-box (SCF) complex containing TIR1) and the auxin or IAA (AUX/IAA) transcription repressors. SCF-TIR1 recruits an E2 ubiquitin conjugating enzyme that then polyubiquitinates AUX/IAAs resulting in rapid degradation by the proteasome. Although all eukaryotes have many forms of SCF in which an F-box protein determines substrate specificity, orthologs of TIR1 and AUX/IAAs are only found in plant species. Thus, the auxin-dependent degradation pathways from plants can be applied, in theory, to other eukaryotic species to induce rapid and reversible depletion of a protein of interest in the presence of auxin. However, the system is generally limited in its application to systems in which homologous recombination is simple and straightforward.

Methods of Tagging a Target Gene Using CRISPR

The present invention provides compositions and methods for tagging a target gene with a degron (e.g., an auxin-inducible degron) in a variety of eukaryotic cells using the CRISPR genome-editing technology. Clustered Regularly Interspaced Short Palindromic Repeats (CRISPR) together with cas (CRISPR-associated) genes was first identified as an adaptive immune system that provides acquired resistance against invading foreign nucleic acids in bacteria and archaea (Barrangou et al. Science 315:1709-12 (2007)). CRISPR consists of arrays of short conserved repeat sequences interspaced by unique variable DNA sequences of similar size called spacers, which often originate from phage or plasmid DNA (Barrangou et al. Science 315:1709-12 (2007); Bolotin et al. Microbiology 151:2551-61 (2005); Mojica et al. J Mol Evol 60:174-82 (2005)). In its native environment, the CRISPR/Cas system functions by acquiring short pieces of foreign DNA (spacers) which are inserted into the CRISPR region and provide immunity against subsequent exposures to phages and plasmids that carry matching sequences (Barrangou et al. Science 315:1709-12 (2007)). The CRISPR/Cas9 system from Streptococcus pyogenes was first characterized as involving only a single gene encoding the Cas9 protein and two RNAs—a mature CRISPR RNA (crRNA) and a partially complementary trans-acting RNA (tracrRNA)—which were identified as necessary and sufficient for RNA-guided silencing of foreign DNAs. Since its discovery, the CRISPR/Cas system has been developed to modify or silence various genes of interest in many organisms. In its most widely used form, Cas9 nuclease is directed by a single guide RNA (sgRNA or guide) to perform site-specific double-strand DNA breaks. Specificity is conferred by complementarity of the sgRNA to the target site in the genome (Cong, L. et al., Science 339, 819-823 (2013); Shalem, O. et al. Science 343, 84-87 (2014); Wang, T. et al. Science 343, 80-84 (2014)).

Thus, using the present invention, a protein of interest (e.g., centromere protein I) can be readily tagged with an AID for rapid degradation in a cell in the presence of auxin and TIR1. Further, this process can be halted and reversed just as rapidly when the system is depleted of auxin and/or TIR1. As those of skill in the art would appreciate, the present methods can be extended to other domains (e.g., degrons) which can be fused to a protein of interest to be targeted for degradation. Like the AUX/IAA-inducible dimerization system of AID/TIR1, other systems capable of promoting recruitment of the tagged protein to an E3 ubiquitin ligase can be used according to the present methods. Some examples of chemically-induced dimerization systems include the pairs FKBP/FRB, FKBP/FKBP, FKBP/CalcineurinA, FKBP/CyP-Fas, GyrB/ByrB, GAI/GID1, and Snap-tag/HaloTag, wherein the dimerization is induced by the agents rapamycin, FK1012, FK506, FKCsA, coumermycin, gibberellin, and HaXS, respectively (Spencer, D M et al., Science 262 (5136): 1019-24, 1993; Ho, S N et al., Nature 382 (6594): 822-6, 1996; Belshaw P J et al., PNAS 93 (10): 4604-7; Rivera et al., Nature Medicine 2 (9): 1028-32, 1996; Farrar, M A et al., Nature 383 (6596): 178-81, 1996; Miyamoto, T et al., Nature chemical biology 8 (5): 465-70, 2012; Erhart, D et al., Chemistry and Biology 20 (4): 549-57, 2013). By way of example, a target gene can be FKBP-tagged at the endogenous locus. An F-box-FRB that binds to the SCF can be expressed such that in the presence of rapamycin, the FKBP-tagged protein is recruited to the SCF for ubiquitination. Accordingly, the present invention contemplates the use of alternative systems that comprise other drug-inducible dimerization systems that promote substrate (e.g., target protein) recruitment to the SCF E3 ligase.

As described herein, an endogenous locus of a target gene can be tagged, e.g., with a degron such as an AID, in a variety of cells (e.g., mammalian cells, insect cells, yeast cells) using the CRISPR genome-editing technology. Thus, in one aspect, the present invention provides a method of tagging a target gene in a cell with a nucleotide sequence encoding an AID.

As used herein, the term “tagging” refers to fusing a target gene sequence in-frame with a sequence encoding a degron—a domain to induce degradation—e.g., an auxin-inducible degron. Typically, tagging allows the degron to be expressed at either the N- or C-terminus of the target protein, thereby labeling the target protein. In some embodiments, the degron is separated from the target protein by other intervening sequences (e.g., the degron sequence does not immediately follow the target protein sequence), as described herein.

Examples of auxin-inducible degrons are known in the art. For example, in some embodiments, an AID used according to the methods of the invention is encoded by a sequence comprising SEQ ID NO: 1. In some embodiments, a portion or a variant of AID that is capable of dimerizing with TIR1 in the presence of auxin can also be used according to the present methods. Briefly, auxins are a major class of plant hormones that influence diverse aspects of plant behavior and development including vascular tissue differentiation, apical development, tropic responses, and organ (e.g., flower, leaf) development. The term “auxin” refers to a diverse group of natural and synthetic chemical substances that are able to stimulate elongation growth in coleoptiles and many stems. Indole-3-acetic acid (IAA) is the principal auxin in higher plants, although other molecules such as 4-chloroindole-3-acetic acid and phenylacetic acid have been shown to have auxin activity. Synthetic auxins include 2,4,5-trichlorophenoxyacetic acid (2,4,5-T) and 2,4-dichlorophenoxyacetic acid (2,4-D).

As used herein, a “target gene” is a nucleotide sequence comprising the sequence encoding the protein to be tagged (e.g., with a degron). “Target gene” includes the nucleotide sequence encoding the protein as well as upstream and downstream non-coding elements (e.g., 5′ and 3′ UTR, and other non-coding regions associated with the coding region of the target gene).

The present method can be practiced using a variety of cells, including, but not limited to, mammalian cells, insect cells, and yeast cells. For example, the present methods can be performed with various cells originating from, e.g., animals, protists, or fungi, such as, for example, a unicellular parasite, a cancer cell, or a non-malignant cell.

Methods of the invention comprise introducing into a cell a nucleic acid comprising a nucleotide sequence encoding a synthetic guide ribonucleic acid (sgRNA), wherein the sgRNA is complementary to a target nucleotide sequence in or near a target gene. Methods of introducing the nucleic acids into cells (e.g., transformation) are known and readily available in the art. See, e.g., Current Protocols in Molecular Biology, Second Edition, Ausubel et al. eds., John Wiley & Sons, 1992; and Molecular Cloning: a Laboratory Manual, 2nd edition, Sambrook et al., 1989, Cold Spring Harbor Laboratory Press.

As used herein, the term “nucleic acid” refers to a polymer comprising multiple nucleotide monomers (e.g., ribonucleotide monomers or deoxyribonucleotide monomers). “Nucleic acid” includes, for example, genomic DNA, cDNA, RNA, and DNA-RNA hybrid molecules. Nucleic acid molecules can be naturally occurring, recombinant, or synthetic. In addition, nucleic acid molecules can be single-stranded, double-stranded or triple-stranded. In some embodiments, nucleic acid molecules can be modified. Nucleic acid modifications include, for example, methylation, substitution of one or more of the naturally occurring nucleotides with a nucleotide analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). “Nucleic acid” does not refer to any particular length of polymer and therefore, can be of substantially any length, typically from about six (6) nucleotides to about 10⁹ nucleotides or larger. In the case of a double-stranded polymer, “nucleic acid” can refer to either or both strands of the molecule.

The term “nucleotide sequence,” in reference to a nucleic acid, refers to a contiguous series of nucleotides that are joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds).

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases (e.g., 2-aminoadenosine, 2-thiothymidine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, 2-thiocytidine).

As used herein, “synthetic guide RNA”, “guide RNA”, and “sgRNA” are used interchangeably. Generally, an sgRNA comprises a nuclease (e.g., Cas9) binding sequence and a targeting sequence that is complementary to a target nucleotide sequence. The nuclease binding sequence in an sgRNA allows for binding of the Cas9 nuclease to form an sgRNA/Cas9 complex. The targeting sequence of the sgRNA directs (guides) the nuclease (e.g., Cas9) to a sequence (target nucleotide sequence) in a genome to allow gene modification via CRISPR.

Generally, for the nuclease (e.g., Cas9) to successfully bind to DNA and modify′ the host genome, the target site is followed by the appropriate protospacer adjacent motif (PAM sequence). The PAM sequence is present in the genomic DNA, but not in the sgRNA sequence. A DNA sequence with the correct target sequence followed by the PAM sequence will be bound by the nuclease. Once bound, the nuclease will cleave the genomic DNA if followed by the appropriate PAM sequence; it the target sequence in the genome is not next to the appropriate PAM sequence, the nuclease does not cleave the genomic DNA. Accordingly, a gene can be modified to remove a PAM sequence to prevent Cas9-mediated cleavage of the genomic DNA, as described herein.

The PAM sequence varies according to the species of the bacteria from which the Cas9 was derived. For example; for Cas9 derived from S. pyogenes, the PAM sequence is NGG located on the immediate 3′ end of the sgRNA targeting sequence (the target sequence in the genome). The PAM sequences of other Cas9 from different bacterial species are known in the art.

The terms “target site” or “target nucleotide sequence” are used interchangeably herein to refer to a nucleic acid sequence present in a target nucleic acid (e.g., a target gene within a genome) that is complementary to, and is bound by, or hybridizes to, a targeting sequence of an sgRNA, provided sufficient conditions for binding exist. In other words, the complement of the target nucleotide sequence has identity to the targeting sequence of an sgRNA. Suitable DNA/RNA binding/hybridizing conditions include physiological conditions normally present in a cell. Other suitable DNA/RNA binding conditions (e.g., conditions in a cell-free system) are known in the art.

Thus, an sgRNA used according to the present methods comprises a targeting sequence that is complementary to a target nucleotide sequence of the host genome (and thus has identity to the complement of the target sequence) that occurs next to an appropriate PAM sequence. As would be appreciated by those of skill in the art, a targeting sequence within a target sgRNA need not be perfectly complementary in sequence to the target sequence (that is, the targeting sequence need not have perfect identity to the complement of the target sequence). In some embodiments, a targeting sequence can have at least about 70%, 75%, 80%, 85%, 90%, 95%, 100%, etc. complementarity to the target nucleotide sequence in the genome of the host that occurs next to an appropriate PAM sequence, provided that the targeting sequence within the sgRNA effects modification of the genome via the CRISPR system. Methods of designing an sgRNA are known in the art.

In certain embodiments, the sgRNA comprises a targeting sequence that is complementary to a nucleotide sequence in the 5′ or 3′ untranslated region (UTR) of the target gene. In other embodiments, the sgRNA comprises a targeting sequence that is complementary to a region in the first or last coding exon of the target gene.

In one embodiment, the nucleic acid comprising a nucleotide sequence encoding an sgRNA is included in a plasmid. In various embodiments, the plasmid contains one or more sequences selected from the group consisting of a promoter sequence, a selection marker sequence, and an inducible recombination sequence. In a particular embodiment, the nucleotide sequence encoding the sgRNA is operably linked to an RNA polymerase III promoter, e.g., a U6 promoter. As used herein, “operably linked” refers to a juxtaposition wherein the components are in a relationship permitting them to function in their intended manner. For example, a promoter is operably linked to a coding sequence if the promoter affects its transcription or expression.

The methods of the present invention further comprise introducing into a cell a nucleic acid comprising a nucleotide sequence encoding a clustered regularly interspaced short palindromic repeat (CRISPR)-associated nuclease 9 (Cas9).

The methods described herein can be carried out using a variety of Cas9 nucleases known in the art, as well as functional variants thereof. As will be apparent to those of skill in the art, Cas9 nucleases that can be used according to the present invention can be that derived from any of a variety of species of bacteria, e.g., Sreptococcus pyogenes or Staphylococcus aureus, and include functional variants of wild-type Cas9, provided the variant is functional as a nuclease. Accordingly, “Cas9” as used herein includes a Cas9 variant comprising a sequence having at least about 40%, 50%, 60%, 70%, 80%, 85%, 90%, 93%, 94%, 95%, 96%, 97%, 98%, or 99% sequence identity to a wild-type Cas9 sequence, or a functional fragment thereof. As used herein, “wild-type” in the context of a Cas9 protein refers to the canonical bacterial amino acid sequence as found in nature (e.g., as occurs in the bacterium S. pyogenes, protein sequence UniProtKB-Q99ZW2 (CAS9_STRP1)).

As used herein, a “fragment” of a Cas9 protein includes any nuclease-active portion of a Cas9 protein. For example, the nucleic acid may encode one or more fragments of Cas9 that retains nuclease activity. (see, e.g., Wright, et al., PNAS, 112(10:2984-89), 2015).

In other examples, a Cas9 variant can possess a nickase activity, also referred to herein as a “Cas9 nickase”. A Cas9 nickase, which can nick one strand of a double-stranded nucleic acid, facilitates homology-directed repair in eukaryotic cells (Cong, et al., Science, 339, 819-23, 2013). A Cas9 nickase can be prepared, for example, by substituting amino acid residues that are required for catalytic activity in a wild-type Cas9 protein with a different amino acid(s). Further, the Cas9 nuclease can be designed to have a relaxed requirement for the Protospacer Adjacent Motif (PAM) sequence (e.g., NGG in S. pyogenes; NNGRRT or NNGRR(N) in S. aureus). Cas9 directs cleavage at sites in the genome which match the appropriate region specified by the sgRNA when they are followed by the PAM sequence. However, modification of key amino acids can confer a relaxed PAM requirement (Heier et al., Nature 519(7542):199-202, 2015). By removing this requirement, the potential targeting applications are greatly increased. Moreover, Cas9 variants designed to recognize different PAMs can also be used in the present methods. Such Cas9 variants prefer PAMs other than the PAMs recognized by their wild-type counterpart, enabling targeting of genes previously not targetable by wild-type Cas9 (Kleinstiver, B P et al. www.ncbi.nlm.nih.gov/pubmed/26098369). Methods of designing, expressing, and testing the functionality of a Cas9 nuclease are routine and known in the art.

The term “sequence identity” means that two nucleotide or amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity or more. For sequence comparison, typically one sequence acts as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In certain embodiments, the nucleotide sequence encoding the Cas9 nuclease or functional variant thereof is codon-optimized. Although the genetic code is degenerate in that most amino acids are represented by several codons (called “synonyms” or “synonymous” codons), it is understood in the art that codon usage by particular organisms is nonrandom and biased towards particular codon triplets. Accordingly, in a particular aspect, the nucleotide sequence encoding a Cas9 nuclease or functional fragment thereof, includes a nucleotide sequence that has been optimized for expression in a particular type of host cell (e.g., through codon optimization). Codon optimization refers to a process in which a polynucleotide encoding a protein of interest is modified to replace particular codons in that polynucleotide with codons that encode the same amino acid(s), but are more commonly used/recognized in the host cell in which the nucleic acid is being expressed. In some aspects, the polynucleotides encoding Cas9 nuclease described herein are codon optimized for expression in mammalian cells.

Upon cleavage of the genomic DNA at the desired target nucleotide sequence by the nuclease (e.g., Cas9), the genomic DNA is appropriately modified using a repair template to insert the desired tag, e.g., AID tag, as well as to, e.g., add, delete, or modify any sequence within the target nucleotide sequence using homology-directed repair (HDR) (see, e.g., Ran et al., Nature Protocols 8(100):2281-308, 2013).

The methods of the present invention rely, in part, on introducing into the cell a repair template comprising a nucleotide sequence encoding an AID. Accordingly, in certain aspects, the present invention also provides a nucleic acid comprising a repair template having a nucleotide sequence encoding an AID.

As used herein, a “repair template” refers to a nucleic acid comprising a nucleotide sequence encoding a degron tag, e.g., an AID tag, and one or more nucleotide sequence that is complementary to one or more portion of a target gene. The term repair template is also referred to in the art as a “donor” or a “rescue” template or construct.

In certain embodiments, the repair template further comprises a heterologous nucleotide sequence operably linked to the nucleotide sequence encoding the AID. For example, the heterologous nucleotide sequence can be selected from the group consisting of a sequence encoding an epitope tag, a sequence encoding a marker protein, a sequence encoding a linker, a promoter sequence, a selection marker sequence, an inducible recombination sequence, and a sequence that replaces a portion of the target gene, or any combination thereof. As such, the heterologous nucleotide sequence included in the repair template can be used to replace one or more nucleotides, introduce one or more additional nucleotides, delete one or more nucleotides, or a combination thereof in the target nucleotide sequences in the cell's genome.

For example, the repair template can be designed to include a nucleotide sequence that replaces a portion of the target gene. In one example, the repair template replaces a PAM in or near the target gene. In another example, the repair template replaces all or a portion of the target nucleotide sequence that is complementary to the targeting sequence of the sgRNA. In these instances, the target gene is tagged with AID, but the target nucleotide sequence is no longer capable of being modified by the CRISPR/Cas9 complex.

In other embodiments, the heterologous nucleotide sequence encodes an amino acid linker that is expressed between the target protein and the AID sequence. In another embodiment, the heterologous nucleotide sequence encodes a fluorescent protein fused to the target protein and the AID, in any desired configuration. In some embodiments, the heterologous sequence is a selection marker sequence that confers antibiotic resistance. Examples of selection markers that can be used in the present methods are known and available in the art. Further, correctly targeted genes can be identified by, e.g., PCR-based strategies or other methods that are known and available in the art.

A repair template typically includes a double-stranded DNA, e.g., a plasmid, a cDNA, a gene block (e.g., gBlocks™ Gene Fragments (IDT)), a PCR product, and the like. In some embodiments, the repair template can include one or more single-stranded portions (e.g., a single-stranded overhang at one or both ends). The size of the repair template can vary and will depend upon the size of the particular nucleotide sequence (e.g., degron tag, heterologous sequence, homology arms, plasmid DNA, etc.) incorporated in a repair template.

In certain embodiments, the repair template may be either in the form of double-stranded DNA, designed similarly to conventional DNA targeting constructs with homology arms (regions of complementarity to the target gene) flanking the insertion sequence, or single-stranded DNA oligonucleotides (ssODNs). The homology arms on each side can vary in length, but are typically longer than 100 bp. For example, the homology arms on each side can be from about 100 bp, about 200, bp, about 300 bp, about 400 bp, about 500 bp, about 600 bp, about 700 bp, about 800 bp, about 900 bp, about 1000 bp, about 1100 bp, about 1200 bo, about 1300 bp, about 1400 bp, or about 1500 bp, etc. This method can be used to generate large modifications, including insertion of reporter genes such as fluorescent proteins or antibiotic resistance markers.

As will be apparent to those of skill in the art, a variety of methods for introducing the nucleic acid components of the present methods into various cells are known and routine in the art. Further, those of skill in the art would recognize that the sgRNA, Cas9, and repair template nucleic acid components of the present method can be introduced into a cell simultaneously, or sequentially, in no particular order. For example, in certain embodiments, nucleic acids can be introduced into a cell that stably expresses a Cas9 nuclease. Thus, the nucleic acid comprising a nucleotide sequence encoding Cas9 could be introduced into the cell at a time earlier than the repair template.

In some embodiments, the cell in which the method is carried out has been modified to express a transport inhibitor response 1 (TIR1) receptor, e.g., an Oryza sativa TIR1 receptor. In certain embodiments, the cell stably expresses the TIR1 receptor (e.g., the nucleotide sequence encoding the TIR1 receptor is stably integrated into the host cell genome).

Genetically-Modified Cells

In further aspects, the present invention also provides genetically-modified cells produced according to the methods described herein. Thus, provided herein is a genetically-modified cell comprising a gene tagged with a nucleotide sequence encoding an auxin-inducible degron (AID). In some embodiments, the gene is an endogenous gene.

In various embodiments, the genetically-modified cell comprising a gene tagged with a nucleotide sequence encoding an AID is a mammalian cell, an insect cell, or a yeast cell. In certain embodiments, the genetically-modified cell originated from, e.g., a cancer cell, or a non-malignant cell (e.g., from a genetic model system).

In some embodiments, the genetically-modified cell further comprises a nucleic acid comprising a nucleotide sequence encoding a transport inhibitor response 1 (TIR1) receptor, e.g., an Oryza sativa TIR1 receptor. In a particular embodiment, the nucleotide sequence encoding the TIR1 receptor is integrated into the genome of the cell.

In another aspect, the present invention provides a genetically-modified cell comprising, a) a nucleic acid comprising a nucleotide sequence encoding Cas9; and b) a nucleic acid comprising a nucleotide sequence encoding a TIR1 receptor. In certain embodiments, the nucleotide sequence encoding the Cas9 nuclease, the nucleotide sequence encoding the TIR1 receptor, or both, is integrated into the genome of the cell. The genetically-modified cell is a mammalian cell, an insect cell, or a yeast cell. In certain embodiments, the genetically-modified cell originated from, e.g., animals, protists, or fungi, and includes any one or more of a unicellular parasite, a cancer cell, or a normal cell from a genetic model system.

In certain embodiments, the Cas9 nuclease is a Streptococcus pyogenes Cas9 nuclease, or a Staphylococcus aureus Cas9 nuclease, or a functional variant or fragments thereof.

In some embodiments, the TIR1 receptor is an Oryza sativa TIR1 receptor.

In additional embodiments, the genetically-modified cell further comprises a nucleic acid comprising a nucleotide sequence encoding a sgRNA. In one embodiment, the nucleic acid comprising the nucleotide sequence encoding the sgRNA can be integrated into the genome of the cell. In one example, the integrated sgRNA can be specific to a target gene within the genome, such that upon expression of Cas9 and the repair template, the sgRNA will direct the Cas9 to the appropriate complementary sequence.

Any one or more of the nucleotide sequences encoding the Cas9 nuclease, the TIR1 receptor, or the sgRNA can operably linked to a heterologous nucleotide sequence. In various embodiments, the heterologous nucleotide sequence is selected from the group consisting of a sequence encoding an epitope tag, a sequence encoding a marker protein, a sequence encoding a linker, a sequence encoding an effector domain, a promoter sequence, a selection marker sequence, an inducible recombination sequence, an inducible promoter sequence, and a locus-targeting sequence, or any combination thereof. Examples of such heterologous sequences are known and routinely used in the art.

Unless defined otherwise, all technical and scientific terms used herein have the same meaning as commonly understood by one of ordinary skill in the art to which this invention pertains.

As used herein, the indefinite articles “a” and “an” should be understood to mean “at least one” unless clearly indicated to the contrary.

The phrase “and/or”, as used herein, should be understood to mean “either or both” of the elements so conjoined, i.e., elements that are conjunctively present in some cases and disjunctively present in other cases.

It should also be understood that, unless clearly indicated to the contrary, in any methods described herein that include more than one step or act, the order of the steps or acts of the method is not necessarily limited to the order in which the steps or acts of the method are recited.

### Exemplification

### CRISPR-Mediated EGFP-AID Tagging of Centromere Protein I

Materials and Methods

Maintenance of Cell Lines

DLD-1 cell lines were cultured as described previously in Dulbecco's Modified Eagle Medium (DMEM) supplemented with 10% fetal bovine serum (FBS), penicillin/streptomycin and 2 mM L-glutamine. Indole-3-acetic acid (IAA) (15148; Sigma) was dissolved in water and added to cells at a concentration of 500 μM for 12 hr.

Plasmids

pX330-GFP was a gift from Chikdu Shivalila and Rudolf Jaenisch (Whitehead Institute/MIT). CRISPR oligos were annealed, phosphorylated and ligated into pX330 as described (Cong, L. et al., Science 339, 819-823, 2013).

C Terminal Tagging

An sgRNA targeting a region in the 3′ UTR, approximately 100 bp downstream of the stop codon was designed using crispr.mit.edu. sgRNAs were designed to contain >3 mismatches to other genomic sequences.

The donor construct for tagging of genes with GFP at the C terminus was originally derived from pL452 (Liu et al., Genome Res. 13:476-484, 2003) and was a gift from Paul Fields and Laurie Boyer (MIT) (McKinley K L and Cheeseman I M, Cell 158:397-411, 2014. The AID-EGFP sequence was amplified from pcDNA5-AID-EGFP and exchanged for GFP in the donor plasmid. This construct contains a 9 amino acid linker between the AID-EGFP and the coding sequence of the target to reduce the chance that the tag will interfere with target function. A region of approximately 1 kb upstream of the stop codon (the 5′ homology arm) and a region of approximately 1 kb after the stop codon (the 3′ homology arm) were amplified from HeLa genomic DNA using iProof (Bio-Rad) or Bio-X-Act DNA polymerases and cloned into the donor plasmid upstream and downstream, respectively, of the AID-EGFP sequence. The stop codon was excluded from the 5′ homology arm to permit an in-frame fusion of the gene and the AID-EGFP sequence. To prevent cutting of the EGFP-AID-tagged allele by SpCas9 (Streptococcus pyogenes Cas9) one of the following two strategies were used: 1) the sgRNA binding site was disrupted by insertion of the EGFP-AID tag; 2) the repair template possessed a mutation in the PAM site.

N Terminal Tagging

An sgRNA targeting a region in the first exon was designed using crispr.mit.edu. sgRNAs were designed to contain >3 mismatches to other genomic sequences.

The sgRNAs was designed to the first exon such that if an allele is cut but not repaired with the template, it has a high likelihood of being repaired by non-homologous end-joining (NHEJ) that generates an indel rendering the allele nonfunctional. Thus, this system can create true replacements (in which no untagged, endogenous alleles remain) either through homology-directed repair (HDR) of both alleles with the tagged template or repair of one allele with the tagged template and knockout of the second allele.

The donor construct for tagging of genes at the N terminus was derived from pL452 (above). The EGFP-AID sequence was amplified from pcDNA5-EGFP-AID and exchanged for GFP in the donor plasmid. This construct contains a 9 amino acid linker between the AID-EGFP and the coding sequence of the target to reduce the chance that the tag will interfere with target function. A region of approximately 1 kb upstream of the start codon (the 5′ homology arm) and a region of approximately 1 kb after the start codon (the 3′ homology arm) were amplified from HeLa genomic DNA using iProof (Bio-Rad) or Bio-X-Act DNA polymerases and cloned into the donor plasmid upstream and downstream, respectively, of the EGFP-AID sequence. To prevent cutting of the EGFP-AID-tagged allele by SpCas9, one of the following two strategies were used: 1) the sgRNA binding site was disrupted by insertion of the EGFP-AID tag; 2) the repair template possessed a mutation in the PAM site.

Generation of Os-TIR1 (Oryza sativa TIR1) Expressing Cells

osTIR1-9Myc was cloned into the pBabe-puro vector and introduced into cell lines using retroviral delivery. Stable integrants were selected in puromycin and single clones isolated using single cell sorting. Western Blot analysis using an antibody raised against the Myc tag was used to determine the expression level of TIR1 protein in different clones. Rapidly growing clones with the high levels of osTIR1 expression were selected for further use.

Generation of AID-Tagged Endogenous Alleles

Two strategies were employed for the recovery of cells that have been cut and repaired with the AID-EGFP template, as detailed below.

Strategy 1

C Terminal Tagging

2.5 ug each of pX330-GFP and the donor plasmid were co-transfected into osTIR1-9Myc expressing cells using Lipofectamine 2000 (Invitrogen) and OptiMEM according to the manufacturer's instructions. 24 h after transfection cells, were transferred to a 20 cm dish, and 48 h later cells were selected in 300 ug G418 (Gibco) and 2 ug/ml puromycin to maintain the osTIR1. Colonies were harvested after 2 weeks and FACS sorted to isolate individual cells.

N Terminal Tagging

The same transfection strategy described above was performed in the absence of selection, as the N terminal construct does not contain a selectable marker. To enrich for transfected cells, cells were sorted for GFP 48 h after transfection with pX330-GFP and the repair construct.

Strategy 2

1 ug of DNA (20:1 molar ratio of a PCR product encoding the repair template and PX459 plasmid containing the sgRNA) were transfected or electroporated into osTIR1-9Myc expressing cells. 24 h later, cells were treated with 2 ug/ml puromycin for 2-3 days to select for transfected cells. After puromycin selection, single clones were isolated by limiting dilution.

Validation of Clones

Clonal populations were screened in one of three ways: 1) Clones were visually inspected for correct localization of the EGFP fusion; 2) PCR to detect the addition of the AID tag; 3) Western Blot to detect the addition of the AID tag.

Results

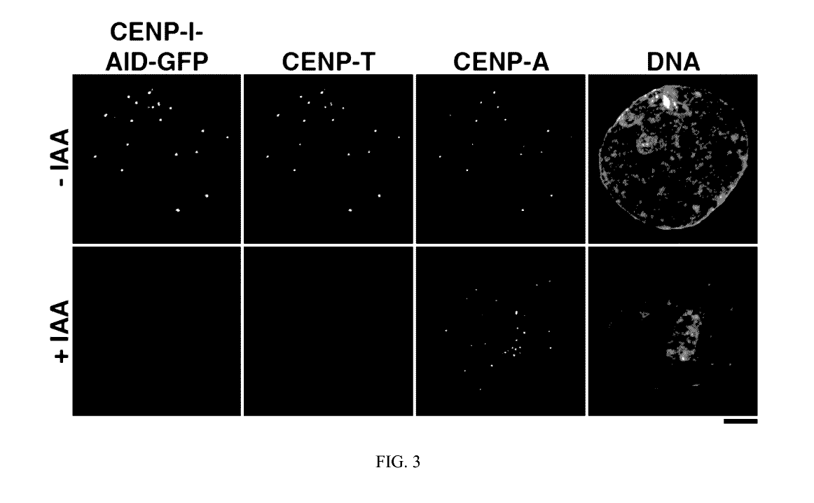

The present study demonstrates successful endogenous tagging of centromere protein I with AID-EGFP using CRISPR. As visualized by immunofluorescence microscopy (FIG. 3), CENP-I-AID-EGFP fusion protein localized to kinetochores in an interphase cell in the absence of IAA. Upon addition of IAA, the fusion protein is no longer observed and associated proteins such as CENP-T are also lost from kinetochores.

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### Auxin-Inducible Degron Sequence

(SEQ ID NO: 1)
atgtccggagccgccgctgctggcggatctGGCAGTGTCGAGCTGAAT
CTGAGGGAGACTGAGCTGTGTCTTGGTCTTCCCGGTGGAGATACAGTG
GCTCCGGTAACCGGAAACAAGAGAGGGTTCTCAGAGACGGTTGATCTG
AAGCTAAATCTGAATAATGAGCCTGCAAACAAGGAAGGATCTACGACT
CATGACGTCGTGACTTTTGATTCCAAGGAGAAGAGTGCTTGTCCTAAA
GATCCAGCCAAACCTCCGGCCAAGGCACAAGTTGTGGGATGGCCACCG
GTGAGATCATACCGGAAGAACGTGATGGTTTCCTGCCAAAAATCAAGC
GGTGGCCCGGAGGCGGCGGCGTTCGTGAAGGTATCAATGGACGGAGCA
CCGTACTTGAGGAAAATCGATTTGAGGATGTATAAAAGCTACGATGAG
CTTTCTAATGCTTTGTCCAACATGTTCAGCTCTTTTACCATGGGCAAA
CATGGAGGAGAAGAAGGAATGATAGACTTCATGAATGAGAGGAAATTG
ATGGATTTGGTGAATAGCTGGGACTATGTTCCCTCTTATGAAGACAAA
GACGGTGATTGGATGCTCGTCGGCGACGTTCCTTGGCCAATGTTCGTC
GATACATGCAAGCGTTTACGTCTCATGAAAGGATCGGATGCCATTGGT
CTCGCTCCGAGGGCGATGGAGAAGTGCAAGAGCAGAGCT

1. A method of tagging a target gene in a cell with a nucleotide sequence encoding an auxin-inducible degron (AID), comprising:
a) introducing into a cell:
1) a nucleic acid comprising a nucleotide sequence encoding a synthetic guide ribonucleic acid (sgRNA), wherein the sgRNA is complementary to a target nucleotide sequence in or near a target gene;
2) a nucleic acid comprising a nucleotide sequence encoding a clustered regularly interspaced short palindromic repeat (CRISPR)-associated nuclease 9 (Cas9);
3) a repair template comprising a nucleotide sequence encoding an AID; and
b) expressing the sgRNA and Cas9 nuclease in the presence of the repair template in the cell,
thereby tagging the target gene with the nucleotide sequence encoding the AID.

2. The method of claim 1, wherein cell has been modified to express a transport inhibitor response 1 (TIR1) receptor.

3. The method of claim 2, wherein the cell stably expresses the TIR1 receptor.

4. (canceled)

5. The method of claim 1, wherein the cell stably expresses the Cas9 nuclease.

6-8. (canceled)

9. The method of claim 1, wherein the repair template further comprises a heterologous nucleotide sequence operably linked to the nucleotide sequence encoding the AID.

10. The method of claim 9, wherein the heterologous nucleotide sequence is selected from the group consisting of a sequence encoding an epitope tag, a sequence encoding a marker protein, a sequence encoding a linker, a promoter sequence, a selection marker sequence, an inducible recombination sequence, and a sequence that replaces a portion of the target gene, or any combination thereof.

11. (canceled)

12. The method of claim 10, wherein the sequence that replaces a portion of the target gene replaces a protospacer adjacent motif (PAM) in or near the target gene.

13-21. (canceled)

22. The method of claim 1, wherein the cell is a mammalian cell, a yeast cell, or an insect cell.

23. A genetically-modified cell comprising,
a) a nucleic acid comprising a nucleotide sequence encoding a clustered regularly interspaced short palindromic repeat (CRISPR)-associated nuclease 9 (Cas9); and
b) a nucleic acid comprising a nucleotide sequence encoding a transport inhibitor response 1 (TIR1) receptor.

24. The genetically-modified cell of claim 23, wherein the nucleotide sequence encoding the Cas9 nuclease, the nucleotide sequence encoding the TIR1 receptor, or both, is integrated into the genome of the cell.

25. The genetically-modified cell of claim 23, further comprising a nucleic acid comprising a nucleotide sequence encoding a synthetic guide ribonucleic acid (sgRNA).

26. (canceled)

27. (canceled)

28. The genetically-modified cell of claim 23, wherein the nucleotide sequence encoding the Cas9 nuclease or the nucleotide sequence encoding the TIR1 receptor, or both, is operably linked to a heterologous nucleotide sequence.

29. (canceled)

30. The genetically-modified cell of claim 25, wherein the nucleic acid comprising the nucleotide sequence encoding the sgRNA is operably linked to a heterologous nucleotide sequence.

31. (canceled)

32. (canceled)

33. A genetically-modified cell comprising a gene tagged with a nucleotide sequence encoding an auxin inducible degron (AID) produced according to the method of claim 1.

34. The genetically-modified cell of claim 33, wherein the cell further comprises a nucleic acid comprising a nucleotide sequence encoding a transport response 1 (TIR1) receptor.

35-37. (canceled)

38. The genetically-modified cell of claim 33, wherein the cell is a mammalian cell, a yeast cell, or an insect cell.

39. A nucleic acid comprising a repair template having a nucleotide sequence encoding an AID.

40. The nucleic acid of claim 39, wherein the repair template is included in a plasmid.

41. (canceled)

42. (canceled)

43. The nucleic acid of claim 39, wherein the repair template further comprises a heterologous nucleotide sequence operably linked to the nucleotide sequence encoding the AID.

44. The nucleic acid of claim 43, wherein the heterologous nucleotide sequence is selected from the group consisting of a sequence encoding an epitope tag, a sequence encoding a marker protein, a sequence encoding a linker, a promoter sequence, a selection marker sequence, an inducible recombination sequence, and a cloning site, or any combination thereof.

45-47. (canceled)


SEQUENCE LISTING (1 sequence)

SEQ ID NO: 1 - DNA, 711 bp, Artificial Sequence
  Auxin-Inducible Degron Sequence
atgtccggagccgccgctgctggcggatctggcagtgtcgagctgaatctgagggagactgagctgtgtcttggtcttcccggtggagatacagtggctccggtaaccggaaacaagagagggttctcagagacggttgatctgaagctaaatctgaataatgagcctgcaaacaaggaaggatctacgactcatgacgtcgtgacttttgattccaaggagaagagtgcttgtcctaaagatccagccaaacctccggccaaggcacaagttgtgggatggccaccggtgagatcataccggaagaacgtgatggtttcctgccaaaaatcaagcggtggcccggaggcggcggcgttcgtgaaggtatcaatggacggagcaccgtacttgaggaaaatcgatttgaggatgtataaaagctacgatgagctttctaatgctttgtccaacatgttcagctcttttaccatgggcaaacatggaggagaagaaggaatgatagacttcatgaatgagaggaaattgatggatttggtgaatagctgggactatgttccctcttatgaagacaaagacggtgattggatgctcgtcggcgacgttccttggccaatgttcgtcgatacatgcaagcgtttacgtctcatgaaaggatcggatgccattggtctcgctccgagggcgatggagaagtgcaagagcagagct